(19) United States
(12) Patent Application Publication
(10) Pub. No.: US 2006/0116592 A1
(43) Pub. Date: Jun. 1, 2006

(54) Method and apparatus for detection and monitoring of T-wave alternans

(75) Inventors: Xiaohong Zhou, Woodbury, MN (US); Thomas J. Mullen, Andover, MN (US); Jeffrey M. Gillberg, Coon Rapids, MN (US); Robert W. Stadler, Shoreview, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 11/186,552

(22) Filed: Jul. 21, 2005

Related U.S. Application Data
(63) Continuation-in-part of application No. 11/000,541, filed on Dec. 1, 2004.

(51) Int. Cl. A61B 5/04

(52) U.S. Cl. 600/509

(57) ABSTRACT

A system and method are provided for assessing T-wave alternans (TWA) using cardiac EGM signals received from implanted electrodes. A T-wave signal parameter is measured from signals received by an automatic gain control sense amplifier. A TWA measurement is computed from a beat-by-beat comparison of T-wave parameter measurements or using frequency spectrum techniques. The TWA measurement magnitude and measurement conditions are used in detecting a clinically important TWA. TWA assessment further includes discriminating concordant and discordant TWA in a multi-vector TWA assessment, and determining the association of a TWA measurement with QRS alternans, mechanical alternans, and other physiological events. A prediction of a pathological cardiac event is made in response to a TWA assessment. A response to a cardiac event prediction is provided.

### RELATED APPLICATION

This application is a continuation-in-part of application Ser. No. 11/000,541, filed Nov. 30, 2004, entitled “Method And Apparatus For Detection and Monitoring of T-Wave Alternans”, incorporated herein by reference in its entirety.

### FIELD OF THE INVENTION

The present invention relates generally to implantable cardiac stimulation/monitoring devices and in particular to an implantable device system and method for assessing T-wave alternans and predicting cardiac events in response to a TWA assessment.

### BACKGROUND OF THE INVENTION

T-wave alternans is a phenomenon observable on surface electrocardiogram (ECG) recordings as a beat-to-beat alternation in the morphology, amplitude, and/or polarity of the T-wave. T-wave alternans (TWA) has been recognized in a variety of clinical conditions, including acquired and congenital long QT syndrome and ischemic heart disease associated with ventricular arrhythmias. TWA is considered an independent predictor for cardiac arrhythmias. Experimentally, TWA has been shown to be a precursor of ventricular tachycardia.

In past practice, TWA has been assessed from surface ECG recordings obtained in a clinical setting. The low-amplitude changes in the T-wave signal during TWA, which is on the order of microvolts, requires complicated software to assess TWA from a surface ECG recording of typically 128 heart beats or more during exercise or high-rate atrial pacing.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a block diagram of an IMD system that may be used for monitoring TWA;

FIG. 2 illustrates one IMD configuration for acquiring EGM data in a TWA assessment method;

FIG. 3 is a flow chart summarizing steps included in a method for collecting EGM data for use in TWA assessment according to one embodiment of the invention;

FIG. 4 is a flow chart summarizing steps included in a method for automatically adjusting EGM sense amplifier gain for obtaining T-wave signals for specialized analysis;

FIG. 5 is a flow chart summarizing steps included in a method for performing signal conditioning and processing operations on the EGM signal data acquired and stored in the signal acquisition method of FIG. 3 and for computing a TWA measurement;

FIG. 6 is a flow chart summarizing steps for evaluating the TWA measurement computed in the method of FIG. 5;

FIG. 7 is a flow chart summarizing steps included in a method for TWA discrimination based on computed TWA measurements;

FIG. 8 is a flow chart summarizing a method that may be used for applying TWA assessment results in managing therapies or predicting pathological cardiac events; and

FIG. 9 is a flow chart summarizing a general method for detecting an alternans pattern in a physiological signal.

### DETAILED DESCRIPTION OF THE INVENTION

The invention provides an implantable medical device system and associated method for monitoring TWA and assessing dynamic changes in TWA for use in tracking disease progression and managing therapies. The system includes an implantable medical device (IMD) capable of monitoring cardiac signals sensed by an associated set of electrodes, a programmer/monitor for interacting with the IMD, and may include an external patient activator. The IMD includes sense amplifiers for receiving cardiac electrogram (EGM) signals from implanted electrodes; signal conditioning circuitry; and a processor for controlling device functions, including EGM signal acquisition and analysis for TWA assessment. The IMD may further include a therapy delivery module for responding to a measurement of TWA predictive of a cardiac event. The external patient activator may be used by the patient or another user to cause the IMD to initiate a TWA monitoring session.

The method for monitoring TWA includes selecting multi-vector EGM sensing electrodes; collecting EGM signals from the multi-vector electrodes at a high heart rate; conditioning the EGM signals wherein signal conditioning steps may include signal deconvolution, data segmentation, noise removal, baseline wander removal, and removal of artificial data; and computing a TWA measurement. The TWA measurement and the measurement conditions (such as heart rate, presence of pacing, and other cardiac mechanical function) are analyzed to determine if a clinically relevant TWA is detected. TWA measurements are further assessed for determining TWA signal consistency and TWA measurement trends for use in predicting cardiac events. TWA assessment can include discriminating between concordant and discordant TWA; discriminating between depolarization/repolarization alternans and repolarization alternans only; and determining association between TWA and mechanical alternans.

TWA measurements may include comparing T-wave amplitudes on consecutive beat pairs to determine if an alternating “A-B-A-B” pattern of a T-wave parameter is present. In alternative embodiments, spectral analysis or other T-wave morphology analysis may be performed in order to identify the presence of T-wave alternans. The TWA measurements may be performed by analysis software included in the implantable device and/or in the external programmer/monitor after downlinking EGM data collected by the IMD to the programmer/monitor for TWA assessment.

A TWA assessment report may be generated and stored in implantable device memory for later transmission to the programmer/monitor. The method may further include evaluating the TWA assessment to determine TWA trends. Based on trend data, a cardiac event may be predicted. Preventative therapies and/or clinician or patient alerts can be delivered in response to a cardiac event prediction. The results of TWA assessment can be used to guide device and/or drug therapy management.

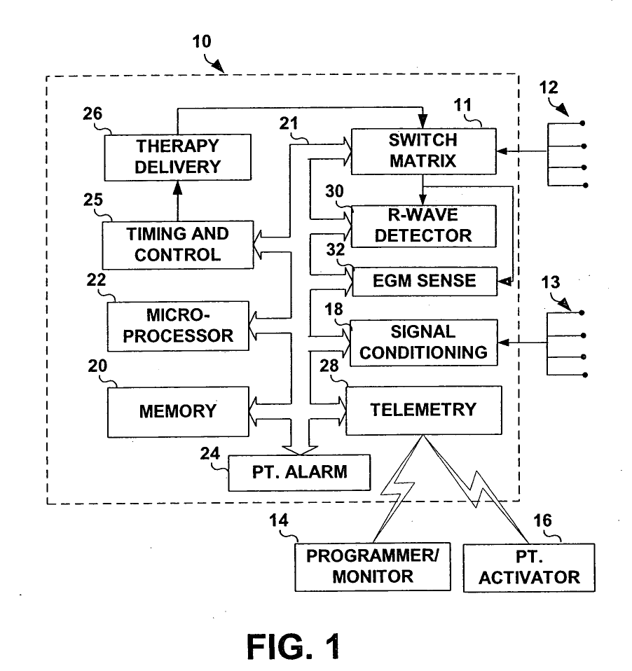

FIG. 1 is a block diagram of an IMD system that may be used for monitoring TWA. The invention provides for dynamic monitoring of TWA in an ambulatory patient. The IMD system includes the IMD 10 and associated electrodes 12 for acquiring EGM signals. EGM signals are used by IMD 10 for assessing cardiac rhythm for determining if and when a therapy is needed. In accordance with the present invention, EGM signals are acquired for TWA assessment.

The IMD 10 may also be coupled to one or more physiological sensors 13, such as an activity sensor or hemodynamic sensors, such as blood pressure sensors. Physiological signals may be used for detecting cardiac events such as arrhythmias or hemodynamic events. Physiological signals may be used by IMD 10 for triggering certain device operations. In one embodiment, physiological signals are used to trigger a TWA assessment.

IMD 10 is adapted for bidirectional communication with an external programmer/monitor 14 via telemetry circuitry 28. Programmer/monitor 14 is used for programming operating parameters in IMD 10 and for downlinking data from IMD 10. In accordance with the present invention, programmer/monitor 14 may be used by a clinician to initiate a TWA assessment. A TWA report may be received by programmer/monitor 14 from IMD 10 including TWA data and/or TWA assessment results. In some embodiments, EGM data acquired by IMD 10 for use in TWA assessment may be transferred to programmer/monitor 14 for analysis by programmer/monitor 14. IMD 10 may also be adapted for communicating with a patient activator 16 which may be used by a patient or other caregiver to initiate a TWA assessment.

IMD 10 includes an R-wave detector 30, which receives EGM signals from electrodes 12 via switch matrix 11. R-wave detector 30 includes a sense amplifier having frequency response characteristics and beat-by-beat automatic adjusting sensitivity for accurate R-wave detection. R-wave detection may generally correspond to that disclosed in U.S. Pat. No. 5,117,824 issued to Keimel et al., U.S. Pat. No. 6,393,316 issued to Gilberg et al., or U.S. Pat. No. 5,312,441 issued to Mader, et al., all of which patents are incorporated herein by reference in their entirety.

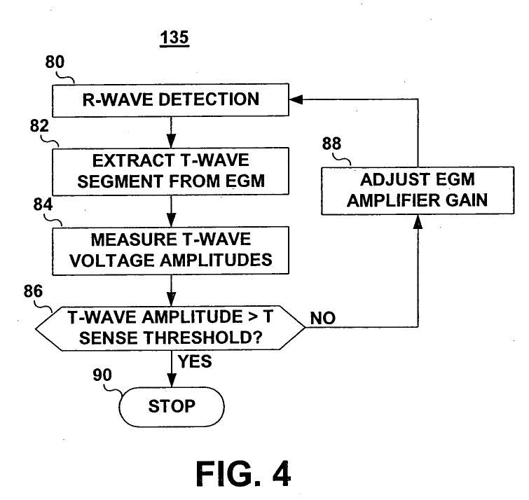

IMD 10 further includes an EGM sense amplifier 32 that may be used for acquiring EGM signals for specialized signal analyses. EGM sense amplifier 32 receives signals from electrodes 12 via switch matrix 11. EGM sense amplifier 32 provides a wider band of frequency response than R-wave detector 30 and a separately adjustable gain setting. In an exemplary embodiment, EGM sense amplifier 32 is embodied as an automatic gain control sense amplifier enabled for automatic gain adjustment responsive to the amplitude of sensed T-wave signals. A method for automatic gain adjustment for T-wave signal analysis will be described below in conjunction with FIG. 4. EGM signal segments for use in specialized analyses may be extracted from EGM signals obtained by sense amplifier 32 based on relative timing from R-waves detected by R-wave detector 30. According to the invention, T-wave signal analysis is performed to obtain T-wave measurements during a T-wave sensing window selected relative to an R-wave detection signal from R-wave detector 30.

Electrodes 12 may be located on leads extending from IMD 10 or may be leadless electrodes incorporated in or on the housing of IMD 10. R-wave detector 30 and EGM sense amplifier 32 receive signals from electrodes 12 via switch matrix 11. Switch matrix 11, under the control of microprocessor 22, is used for selecting which electrodes are coupled to R-wave detector 30 for reliable R-wave detection and which electrodes are coupled to EGM sense amplifier 32 for use in TWA assessment.

IMD 10 includes a signal conditioning module 18 for receiving EGM signals from EGM sense amplifier 32 and physiological signals from sensors 13. Signal conditioning module 18 includes sense amplifiers and may include other signal conditioning circuitry such as filters and an analog-to-digital converter. Microprocessor 22 receives signals from signal conditioning module 18 for detecting physiological events.

Memory 20 is provided for storing conditioned EGM signal output from conditioning module 18. In one embodiment, processing of EGM signals for assessing TWA is performed by IMD microprocessor 22. Microprocessor 22, controls IMD functions according to algorithms and operating parameters stored in memory 20. Microprocessor 22 may perform TWA assessment according to the methods to be described below. In response to TWA assessment results, microprocessor 22 may cause an alert signal to be generated by alarm circuitry 24. Additionally or alternatively, a therapy delivery module 26 may be signaled to deliver or withhold a therapy, or adjust therapy delivery parameters under the control of timing and control circuitry 25.

In other embodiments, EGM data acquired by IMD 10 for use in TWA assessment may be stored in memory 20 and downlinked to external programmer/monitor 14. Processing circuitry included in programmer/monitor 14 may then perform a TWA assessment according to programmed-in algorithms. Reports of TWA assessment results may be generated by either IMD 10 or external programmer/monitor 14, for display, printing or electronic storage such that the results are available for review by a clinician.

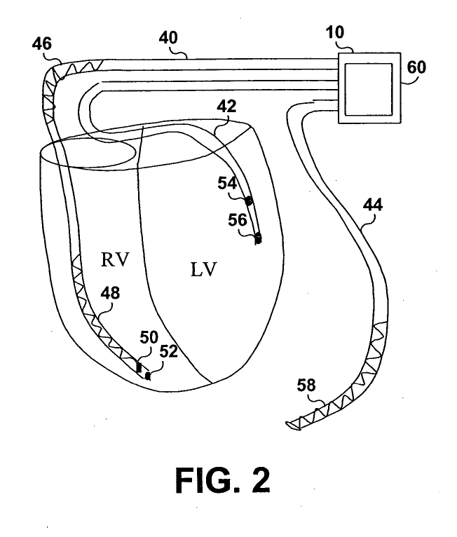

FIG. 2 illustrates one IMD configuration for acquiring EGM data in a TWA assessment method. IMD 10 may be embodied as any of a number of IMDs, such as a cardiac monitoring device, a pacemaker, an implantable cardioverter defibrillator, a neurostimulator, or a drug delivery device. EGM data suitable for assessing TWA may be acquired from signals sensed by subcutaneous electrodes, epicardial electrodes, transvenous or endocardial electrodes, or a neurostimulation lead. In an exemplary embodiment, multiple sensing vectors are selected for acquiring EGM data for TWA assessment. Multiple sensing vectors may be selected from any combination of available electrodes.

In the example shown in FIG. 2, IMD 10 is embodied as an implantable cardioverter defibrillator and is shown coupled to a set of leads adapted for delivering pacing, cardioversion, and defibrillation pulses and sensing EGM signals for detecting and discriminating heart rhythms. IMD 10 is coupled to a right ventricular (RV) lead 40 carrying a superior vena cava (SVC) coil electrode 46 and an RV coil electrode 48 for use in delivering cardioversion and defibrillation shock pulses. RV lead 40 carries a tip electrode 52 and a ring electrode 50 used in pacing and sensing functions in the right ventricle.

IMD 10 is further coupled to a coronary sinus (CS) lead 42 equipped with a tip electrode 56 and ring electrode 54 for use in sensing and pacing functions in the left heart chambers. CS lead 42 may be advanced into a cardiac vein so as to position CS tip electrode 56 and ring electrode 54 in a desired location over the left ventricle.

IMD 10 is provided with a can or case electrode 60 that may be used in combination with any of the cardiac electrodes for delivering stimulation pulses or sensing cardiac electrical signals in a unipolar mode. IMD 10 may be coupled to one or more subcutaneous leads 44 carrying a subcutaneous electrode 58, which may be a coil, patch or other type of electrode used in combination with SVC coil electrode 46, RV coil electrode 48, and/or can electrode 60 for delivering cardioversion or defibrillation shock pulses. Subcutaneous electrode 58 may alternatively be used in combination with any of the tip or ring electrodes 50, 52, 54 and 56 for sensing or pacing in unipolar modes.

Numerous sensing vectors may be selected from the electrodes available in the system shown in FIG. 2. Any electrode located on RV lead 40 or CS lead 42 may be selected in a unipolar sensing combination with can electrode 60 or subcutaneous electrode 58. Any combination of two electrodes located on RV lead 40 or CS lead 42 may be selected for bipolar sensing. Thus multi-vector sensing for TWA assessment may be achieved by selecting multiple unipolar and/or bipolar sensing electrode pairs, either simultaneously or sequentially, for collecting EGM signals. Both far-field and near-field EGM signals can be collected for TWA assessment. Multi-vector TWA analysis allows discrimination of concordant and discordant forms of TWA. The invention is not limited to the lead and electrode arrangement shown in FIG. 2. Numerous variations exist in the types of leads and electrodes that may be included in a system for monitoring TWA.

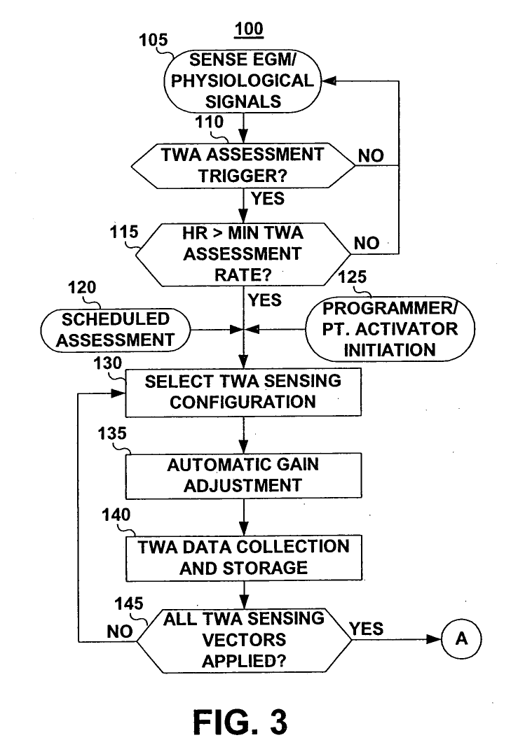

FIG. 3 is a flow chart summarizing steps included in a method for collecting EGM data for use in TWA assessment according to one embodiment of the invention. At step 105, cardiac EGM signals and any other physiological sensed signals are collected by the IMD. These signals may be monitored under normal IMD operating conditions, for example for determining when a pacing or arrhythmia therapy or other therapy delivery is needed. For the purposes of the present invention, one or more physiological signals may be used in determining when a TWA assessment should be initiated.

A number of conditions may be defined as triggering conditions for a TWA assessment. Detection of a TWA assessment trigger condition is determined at decision step 110 based on monitored EGM and/or other physiological signals. Physiological events thought to have a causal relationship or other correlation to the occurrence of TWA may be specified as TWA assessment triggering events, thereby facilitating an evaluation of the association between the physiological events and TWA. For example, detection of an elevated heart rate that is greater than some predefined rate may trigger TWA assessment. Other physiological conditions that may trigger a TWA assessment may include detection of increased activity based on an activity sensor, a change in a hemodynamic signal such as blood pressure, or detection of a premature ventricular contraction (PVC) or other arrhythmia.

In one embodiment, detection of a PVC initiates a beat-to-beat T-wave alternans assessment. An increased magnitude of beat-to-beat TWA may be used to predict an imminent occurrence of ventricular tachyarrhythmias or represent deterioration of ventricular function. A beat-to-beat TWA assessment may be performed using T-wave signals acquired from a relatively short series of beats, for example 10 to 20 beats, following the PVC.

Method 100 continues sensing EGM and other physiological signals (step 105) until a physiological trigger condition is detected at step 110. Once a TWA assessment trigger is detected, method 100 determines if the current heart rate is greater than a TWA assessment minimum rate. Typically, TWA is not present or is not measurable at low or resting heart rates. As such, a minimum heart rate, for example 80 bpm, may be selected as a required condition before initiating a TWA assessment. If the heart rate is below the minimum TWA assessment rate, method 100 may return to step 105 and continue monitoring EGM and physiological signals until the heart rate reaches the required rate.

In some embodiments, TWA assessment may be performed on a scheduled basis, e.g., hourly, daily, weekly or otherwise. In method 100, TWA assessment initiated on a scheduled basis is indicated by step 120. As described previously, TWA assessment may be initiated by the patient or another caregiver using a programmer or a patient activator. Initiation of a TWA assessment using a programmer or patient activator is indicated by step 125.

When a scheduled TWA assessment is performed, or when a TWA assessment is triggered by a patient activator or programmer, the TWA assessment will typically include pacing at a rate expected to provoke a measurable TWA pattern. The pacing rate may be, for example, in the range of 80 to 120 bpm. In some embodiments, a condition that causes pacing at a high rate, such as detection of increased activity or metabolic demand, may initiate a TWA assessment. Pacing may be single, dual or multi-chamber pacing. When a TWA assessment that includes pacing at a high rate is triggered, step 115 for verifying that the heart rate is greater than a minimum assessment rate is unnecessary.

Once all conditions are met for performing a TWA assessment, a TWA electrode sensing configuration is selected at step 130. The configuration selected will depend on the IMD system used. In an exemplary embodiment, multiple sensing vectors are selected for acquiring EGM data for TWA assessment. Depending on the IMD sensing capabilities, the multiple sensing vectors may be selected individually in a sequential manner. If the IMD allows for multiple EGM signals to be acquired simultaneously, multiple sensing vectors may be selected for simultaneous EGM sensing. An implantable cardioverter defibrillator may be capable of acquiring two or more EGM signals at a time. As such, two or more sensing vectors may be selected simultaneously for acquiring EGM signals for use in TWA assessment. Additional sensing vectors may be selected in sequential pairs for obtaining additional EGM signals for use in TWA assessment. In alternative embodiments, a sensing configuration for acquiring EGM signals for TWA may be programmed by a clinician.

In the example electrode arrangement shown in FIG. 2, some of the sensing vectors that may be selected for TWA assessment are: RV tip electrode 52 to RV ring electrode 50, RV tip electrode 52 to can electrode 60, CS tip electrode 56 to CS ring electrode 54, CS tip electrode 56 to can electrode 60, RV coil electrode 48 to can electrode 60, SVC coil electrode 46 to can electrode 60, and subcutaneous electrode 58 to can electrode 60. Unipolar sensing vectors will generally include both near-field and far-field signal information for global TWA measurements. Bipolar sensing vectors will generally include near-field signal information for local TWA measurements.

At step 135, automatic gain adjustment is performed. As indicated previously, an EGM sense amplifier included in the IMD is an automatic gain control amplifier. As such, if the T-wave amplitude does not exceed a T-wave sensing threshold, the sense amplifier gain is automatically adjusted at step 135. A method for automatic gain adjustment for T-wave sensing will be described below in conjunction with FIG. 4. Alternatively, the clinician may program a selected sensing vector and a corresponding amplifier gain.

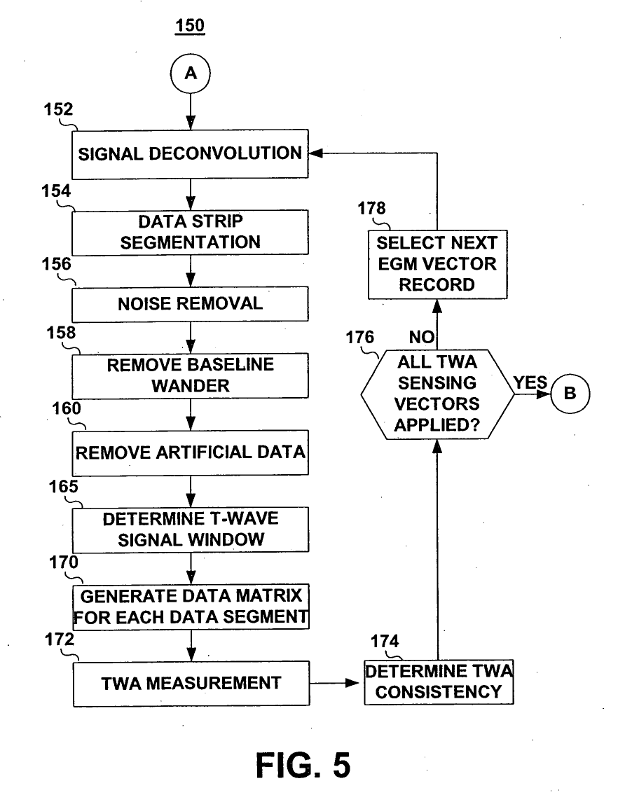

At step 140, data for assessing TWA is collected and stored. EGM signals for each sensing vector may be acquired for several seconds or minutes. The selected EGM signal(s) are stored in memory for use by processing circuitry in a TWA measurement method 150 to be described below in conjunction with FIG. 5. The TWA measurement method evaluates the T-wave signals included in the EGM data stored at step 140. Other signals may be acquired at step 140 for use in a TWA assessment. In order to ensure a reliable measurement of TWA, the EGM signals acquired at step 140 may be evaluated for the presence of signals other than T-wave signals. For example, the EGM signal may be evaluated for R-wave alternans, premature contractions or other conduction aberrancies as well as electromagnetic interference or other signal noise.

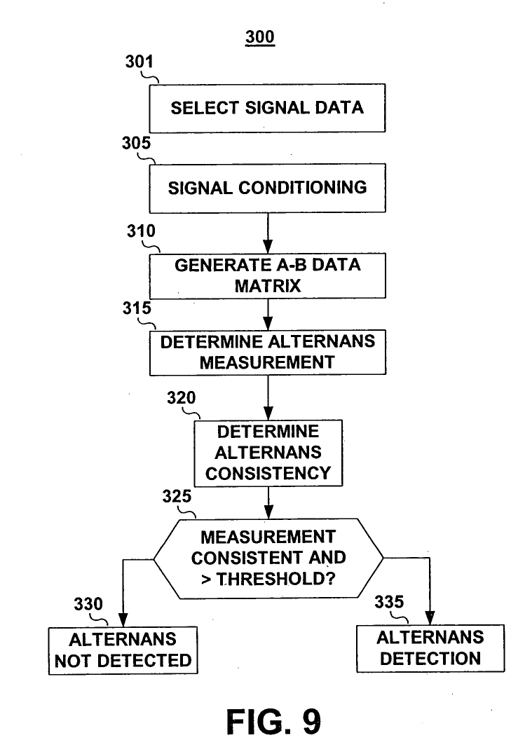

The presence of mechanical alternans or hemodynamic dysfunction associated with the presence of TWA may be clinically relevant in predicting cardiac events or diagnosing a deteriorating cardiac condition. Therefore, other physiological signals may also be acquired at step 140 that relate to the mechanical function of the heart. Signals useful for detecting the presence of mechanical alternans or hemodynamic dysfunction include, for example, a blood pressure signal or a wall motion signal obtained from physiological sensors. Such signals may be evaluated to allow better interpretation of the TWA measurements. Mechanical alternans and R-wave alternans may be determined according to a general method described below in conjunction with FIG. 9.

Method 100 returns to step 130 to select the next TWA sensing configuration if EGM signals have not yet been obtained from each of the desired sensing vectors, as determined at decision step 145. If all sensing vectors have been applied, method 100 proceeds to method 150 in FIG. 5 for signal conditioning and processing.

FIG. 4 is a flow chart summarizing steps included in a method for automatically adjusting EGM sense amplifier gain for obtaining T-wave signals for specialized analysis. The method shown in FIG. 4 represents a subroutine that may be performed at step 135 for automatic gain adjustment in method 100 of FIG. 3. At step 80, an R-wave is detected from a sensed EGM signal using any known R-wave detection circuitry and method. A timing signal from the R-wave detector 30 (shown in FIG. 1) can be used to blank out or exclude the QRS signal from a separate EGM signal obtained by EGM sensor 30 (FIG. 1), leaving the T-wave portion of the EGM signal to be analyzed for adjusting the gain. Thus, at step 82, the T-wave segment is extracted from the EGM signal by removal of the QRS segment according to the timing of R-wave detection.

At step 84, the EGM signal voltages in the extracted T-wave segment are analyzed. If the signal voltage exceeds a predefined T-wave sensing threshold, no adjustment is made to the sense amplifier gain. If the signal voltage amplitude does not exceed the predefined threshold, the EGM sense amplifier gain is increased. Amplifier gain is increased until the extracted T-wave segment signal voltages exceed a predetermined sensing threshold. In one embodiment, EGM sense amplifier gain is increased to ensure that a certain percentage (e.g., 75%) of the dynamic range of the system is utilized to maximize signal resolution, while preventing signal clipping. During automatic gain adjustment for T-wave sensing, the gain of the sense amplifier included in the R-wave detector is unchanged so that accurate R-wave detection continues without saturation of the QRS signal.

FIG. 5 is a flow chart summarizing steps included in a method for performing signal conditioning and processing operations on the EGM signal data acquired and stored in method 100 of FIG. 3. Steps 152 through 160 shown in FIG. 5 include signal conditioning steps that are performed to improve the T-wave signal-to-noise ratio. Steps 152 through 160 include representative signal conditioning steps, all of which may or may not be needed to achieve acceptable signal-to-noise ratio. Signal conditioning steps implemented for improving signal-to-noise ratio will depend in part on the signal acquisition conditions and may also depend on the T-wave measurements that will be made for assessing TWA.

Step 152 represents a signal deconvolution step which may be required when EGM signals are acquired using a high-pass filter. The QRS complex can be obtained using high-pass filtered signals, however the T-wave is of lower frequency than the R-wave. If the EGM signals are obtained using a high-pass filter, for example a filter that passes signals greater than about 0.5 Hz, signal deconvolution step 152 may be used to inversely convert 5 Hz signals to 0.05 Hz signals.

At step 154, stored EGM records are segmented into strips. EGM records stored for each sensing vector may be several minutes, or even 10 minutes or more, in length. In one embodiment, TWA analysis is performed on segmented EGM records. Each segment represents a window of time, and TWA measurements may be performed using averaging, subtraction or spectral analysis techniques over each time window as will be described in greater detail below. For example, EGM records several minutes in length may be segmented into strips of about 20 seconds in length. Depending on the length of EGM records and the methods used to perform TWA measurements, this segmentation step may not be necessary but can be useful in making data analysis steps more manageable. Averaging T-wave parameters used in making a TWA measurement over segmented data records may also reduce the variation of the TWA measurements.

At step 156 EGM signal noise is removed. Noise removal may be performed using standard analog or digital filtering methods, for example an N^{th} order digital Butterworth filter may be used to remove EGM signal noise. In one embodiment, an 8^{th} order digital Butterworth filter is used to remove EGM signal noise.

At step 158 baseline wander is removed. One method for removing baseline wander utilizes cubic Hermite line methods. Other baseline correction tools may be used.

At step 160, artificial data is removed. Artificial data may be present due to the occurrence of PVCs or other artifacts that are not true ORS and T-wave events. PVC detection methods may be used for removing signals associated with PVCs that may obscure TWA measurements. PVC detection is typically based on the detection of two consecutive R-waves without detection of an intervening atrial event (P-wave). Template matching of R-wave signals may be used to identify normal beats and exclude abnormalities associated with slow VT, runs of PVC, or aberrant conduction if it is determined that aberrancy affects TWA measurements. A template matching method that may be adapted for use with the present invention for identifying normal R-wave signals is generally disclosed in the above-reference Gillberg, patent. When a T-wave signal is removed as artificial data, the succeeding T-wave may also be removed in order to maintain an A-B-A-B T-wave pattern. Alternatively, a removed T-wave signal may be replaced by an average of a previous number of respective “A” or “B” T-waves so that the A-B pattern will remain.

At step 165, a T-wave signal window location is determined. The T-wave will occur during a window of time following a QRS complex. The beginning of a QRS complex can be a ventricular sensing or a ventricular pacing marker. At step 165, temporal characteristics of the EGM signal during a single beat are determined to allow the T-wave to be correctly identified and a T-wave parameter measured for TWA assessment. In one embodiment, the QRS duration and the S-T interval are determined.

The QRS duration may be measured from the intrinsic EGM signal. The QRS duration may be measured starting at a point defined by dV/dtmax on the QRS complex, a threshold crossing, or other defined QRS starting point. The end of the QRS complex may be defined as some threshold crossing, dV/dtmin, or a zero-crossing. Within the QRS duration the amplitude will be determined so that an alternans in QRS duration and amplitude can be assessed for determining if QRS alternans (depolarization alternans) is related to TWA (repolarization alternans) or exists alone.

The point defined as the end of the QRS complex and the point defining the start of the subsequent T-wave are used to measure the S-T interval. The start of the subsequent T-wave may be determined as a threshold crossing, dV/dtmax, or other feature identifiable on the T-wave. Using the QRS width and S-T interval, the start of a T-wave signal window may be calculated relative to the start of the QRS signal.

Once the T-wave signal window location is determined, a beat-to-beat TWA analysis may be performed by generating a data matrix for each data segment at step 170. Data matrix formation includes assigning every other T-wave an “A” label and intervening T-waves a “B” label. T-wave measurements corresponding to “A” and “B” labeled T-waves are then stored in the data matrix. In one embodiment, T-wave amplitudes are measured and a matrix of “A” T-wave amplitudes and “B” T-wave amplitudes is generated. T-wave amplitudes may be measured as an average signal voltage, a peak voltage, or a peak-to-peak voltage difference.

In other embodiments, other T-wave parameters may be measured for generating the data matrix at step 170. Morphological features could be determined such as a T-wave template, T-wave width at a given threshold crossing, or other features that allow TWA to be distinguished by measuring consistent differences between “A” and “B” T-waves. Spectral analysis may alternatively be performed in which frequency-domain measurements are used in generating the data matrix for “A” and “B” labeled T-waves. Any T-wave parameter that allows the A-B-A-B-A-B pattern of TWA to be ascertained may be measured at step 170.

At step 172, TWA measurements are determined by comparative analysis of the “A” and “B” labeled T-wave measurements stored in the data matrix generated in previous step 170. Measurements may be compared on a beat-to-beat basis to determine the difference between “A” labeled T-wave measurements and “B” labeled T-wave measurements. In the example given above in which T-wave amplitude measurements are stored, the beat-to-beat amplitude difference between “A” labeled T-waves and “B” labeled T-waves is calculated. The TWA measurement obtained at step 172 could then be computed as the average of the differences between the “A” and “B” T-wave pairs. Differences may be averaged over each data segment and an overall average may be computed from the segment averages or from the beat-to-beat differences.

Alternatively or additionally, T-wave measurements may be averaged over each data segment for the respective “A” and “B” labeled measurements. The difference between the averaged “A” measurement and the averaged “B” measurement may then be determined. In the example of T-wave amplitude measurements, all “A” amplitudes may be averaged to determine a mean “A” T-wave amplitude. All “B” amplitudes may be averaged to determine a mean “B” T-wave amplitude. The TWA measurement determined at step 172 would then be computed as the difference between the average “A” T-wave amplitude and the average “B” T-wave amplitude. The TWA measurement for each data segment may be averaged over an entire EGM record.

The operations performed at step 172 may therefore include determining differences in T-wave parameters between “A” and “B” beats on a beat-by-beat basis and further performing statistical analysis on the differences to determine an overall TWA measurement parameter. Alternatively, statistical analyses may be performed on the “A” and “B” T-wave parameters first to determine mean “A” and mean “B” T-wave parameters. The difference between the means may then be used to compute an overall TWA measurement parameter.

At step 172, TWA assessment can alternatively be performed using spectral analysis of a time series of T-wave parameters rather than a beat-by-beat comparison. The amplitude at a selected time point on the T-wave is measured for a series of T-waves. The measured amplitudes forms a time series. The power spectrum of this time series is then calculated using Fourier Transform methods to determine if an alternans pattern is present as evidenced by two substantially equal dominant frequency peaks.

At step 174, the TWA measurement is evaluated for possible contamination due to artifacts or signal noise. This evaluation is based on the differences between “A” and “B” T-waves and artifacts occurring in the T-wave signals. If TWA is present, the differences in the “A” and “B” T-waves will be consistent in phase evidencing an A-B-A-B-A-B pattern. For example, if T-wave amplitudes are measured, the “A” T-wave amplitudes will be greater than the “B” T-wave amplitudes most of the time or less than the “B” T-wave amplitudes most of the time. Considerable variation in the comparative relation of the “A” and “B” T-waves does not evidence an alternans pattern. At step 174, method 150 verifies that the beat-to-beat differences between “A” and “B” T-wave parameters are consistent in phase. If the differences are changing in phase, i.e., “A” measurements are sometimes greater and sometimes less than “B” measurements, the TWA measurement may not be considered clinically significant. The TWA consistency may be evaluated at step 174 by determining the percentage of all beat-to-beat differences being of the same phase.

Determination of TWA consistency at step 174 may include a determination of the frequency of PVCs and the frequency of T-wave artifacts in the acquired EGM signals. For example, when PVCs and T-wave artifacts occur in greater than a predetermined percentage of the T-wave cycles, for example greater than 15% of the T-wave cycles, the TWA measurement may not be representative of a true TWA and therefore not have clinical meaning. Determination of TWA consistency may also include a determination of the contribution of respiratory activity to T-wave signal variation and the net effect on the TWA measurement.

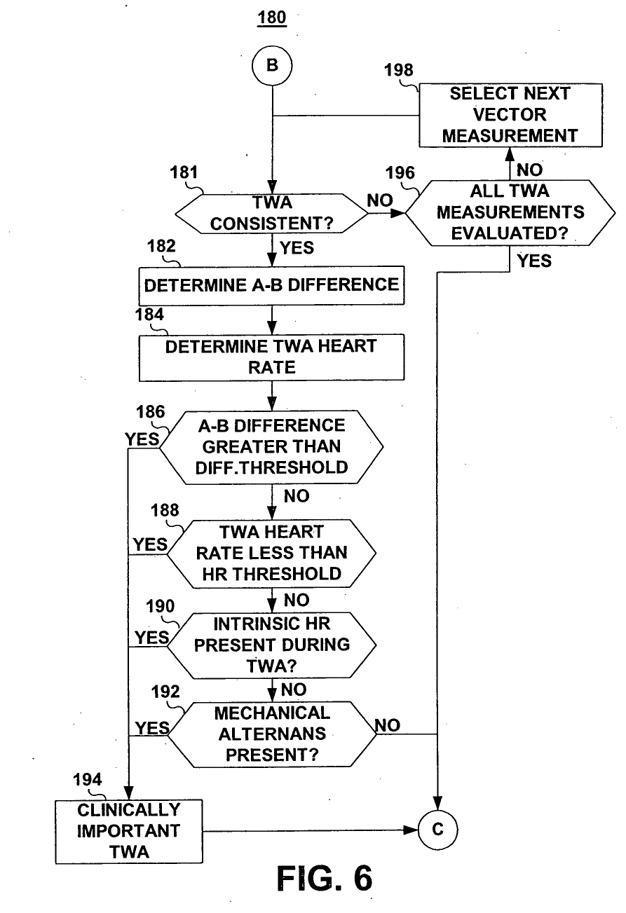

At step 176, method 150 determines if TWA measurements have been computed for all of the acquired EGM vector records. If not, the next EGM vector record is selected at step 178, and method 150 is repeated. Once a TWA measurement has been computed for each of the EGM vectors acquired, method 150 proceeds to method 180 shown in FIG. 6 for evaluating the clinical significance of the TWA measurement. If the TWA sensing electrode configurations for use during TWA assessment are programmed by a clinician method 150 will be repeated only for the specifically programmed sensing configurations.

FIG. 6 is a flow chart summarizing steps for evaluating the TWA measurement computed in the method of FIG. 5. A TWA measurement may or may not have clinical significance depending on the magnitude of the measurement and the conditions under which the TWA was provoked. Steps shown in FIG. 6 present an evaluation of the TWA measurement that may be performed for assessing the seriousness of the measurement. In some embodiments, the TWA measurements may be reported for evaluation by a clinician, without further evaluation by the IMD system as shown in FIG. 6.

At decision step 181, the consistency of the TWA signal is verified. If the TWA signal is determined to be inconsistent, according to the result of step 174 of method 150 (FIG. 5), the TWA measurement may be concluded to be clinically insignificant. If all TWA measurements have not yet been evaluated, as determined at decision step 196, the TWA measurement associated with the next vector of a multi-vector analysis is selected at step 198. If the alternans pattern was determined to be consistent, the TWA measurement and conditions under which the TWA was present are evaluated to determine the clinical significance of the TWA.

At step 182, a TWA parameter used to determine the TWA measurement is determined. The TWA parameter may be a difference between an “A” and “B” T-wave measurements or an alternans power/voltage determined from spectral analysis. The TWA parameter determined at step 182 may be equivalent to the TWA measurement determined at step 172 in method 150 or an intermediate result. The heart rate or pacing rate during the TWA measurement is determined at step 184. The heart rate may be determined from the R-wave detection rate during EGM signal acquisition or computed from the EGM signal used for TWA assessment. Both the magnitude of TWA parameters and the heart rate at which TWA occurs can indicate the severity of the TWA in terms of predicting a cardiac event or diagnosing a worsening cardiac condition.

At decision step 186, TWA parameter(s) are compared to a predetermined threshold or other criteria set for indicating the severity of the TWA based on the A-B difference or alternans power/voltage. If the magnitude of the difference or alternans power/voltage exceeds the threshold, the TWA is flagged as clinically important at step 194.

At decision step 188, the heart rate at which the TWA was measured is compared to a predetermined heart rate (HR) threshold. If the heart rate is slower than a predetermined threshold rate, the TWA is flagged as clinically important at step 194. An A-B difference threshold may be set for different heart rate ranges for determining when the TWA measurement is considered clinically important.

TWA that is present during an intrinsic rhythm is likely to be more serious than TWA provoked during pacing. At decision step 190, a determination is made whether the TWA measurement occurred during pacing or intrinsic rhythm. If the TWA measurement is associated with an intrinsic rhythm, the measurement is flagged as clinically important at step 194.

If TWA is accompanied by mechanical alternans, the TWA may be associated with worsening cardiac dysfunction. At decision step 192, a determination is made whether the TWA measurement is associated with the presence of mechanical alternans. If mechanical alternans is concomitant with TWA, the TWA measurement is flagged as clinically important at step 194. Mechanical alternans is detected by evaluating a hemodynamic or mechanical cardiac signal, such as blood pressure, wall motion, blood flow, or chamber volume. A general method for detecting an alternans pattern from a physiological signal is described below in conjunction with FIG. 9.

Decision steps 186 through 192 are shown as exclusive steps in method 180 such that if any one condition is satisfied the TWA measurement is flagged as clinically important. It is recognized that conditions for determining the clinical significance of a TWA measurement may not be mutually exclusive. As noted previously, the magnitude of the A-B difference that is considered clinically important may depend on the paced or intrinsic heart rate. Therefore, a combination of criteria, not limited to the criteria listed in method 180, may be defined for determining the clinical importance of the TWA measurement.

Thresholds or other criteria used in identifying clinically significant TWA measurements may be updated over time by a clinician based on individual patient need or automatically through a learning process. An automated learning process updates thresholds or other criteria defining clinically important TWA measurements based on the correlation of TWA measurements with other physiological signals or cardiac events.

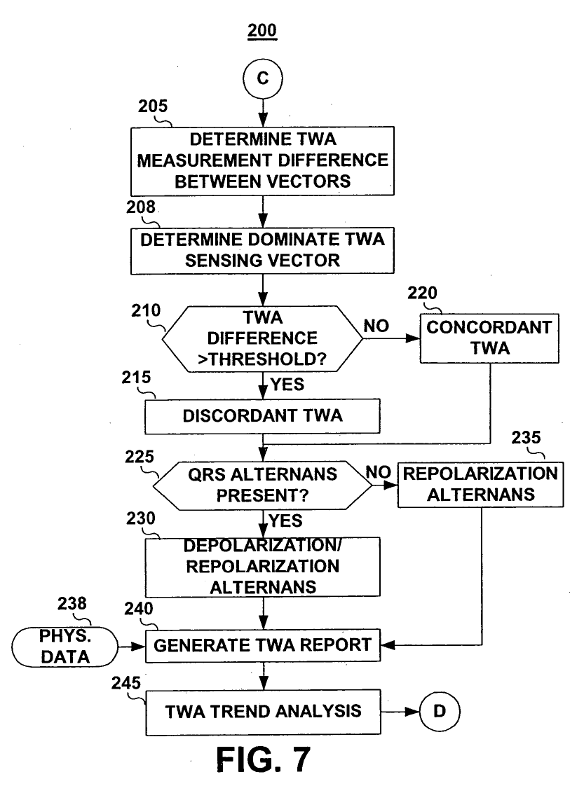

Method 180 is repeated for each of the TWA measurements obtained from multiple sensing vectors. Alternatively, method 180 may be performed only for the vector producing a maximum TWA measurement, referred to herein as the “dominant” TWA sensing vector. After completing the comparative analyses provided in method 180, further assessment of TWA can be performed according to method 200 shown in FIG. 7.

FIG. 7 is a flow chart summarizing steps included in a method for TWA discrimination based on the TWA measurements determined in method 150 of FIG. 5. At step 205, differences between the TWA measurements obtained for each of the EGM vector records is determined. The dominant TWA sensing vector, i.e., the EGM sensing vector producing the maximum TWA measurement will be determined at step 208. The TWA measurement differences are compared to a threshold at decision step 210. If differences exist in the manifestation of TWA as measured by different sensing vectors, in particular measurements made from different near-field signals obtained from local ventricular regions, discordant TWA is present. Discordant TWA is considered a more serious condition than concordant TWA in that discordant TWA may be more arrhythmogenic than concordant TWA.

If the TWA measurement is the average difference between “A” and “B” T-wave amplitudes, the difference between the average difference determined for one EGM sensing vector and the average difference determined for another EGM sensing vector is determined at step 205. If the difference between vectors is greater than some predefined threshold, then discordant TWA is present as concluded at step 215. If the difference is less than some predefined threshold, then concordant TWA is present as concluded at step 220.

At decision step 225, method 200 determines if QRS alternans is present. QRS alternans may be determined using methods generally described below in conjunction with FIG. 9. QRS alternans can be present in R-wave amplitude, QRS width, and/or signal frequency. ORS signals from recorded EGM signals are evaluated to determine if a QRS parameter such as R-wave amplitude, varies in an alternating beat-to-beat manner. If QRS alternans is present, depolarization and repolarization alternans is present as concluded at step 230. This result may be clinically meaningful in that the TWA may be present as a result of the QRS alternans and therefore treatment options may be different. If QRS alternans is not present, only repolarization alternans is present as concluded at step 235.

At step 240 a TWA assessment report is generated. The report may be stored in IMD memory and available for later downlinking to a programmer/monitor. In some embodiments, the TWA assessment computations may be performed by an external programmer/monitor or other computer and the generated report may be made available for immediate display, printing or electronic storage. The report may include a number of results and conclusions determined from the TWA assessment.

In one embodiment, the report includes the resulting TWA measurements for each sensing vector in a multi-vector TWA assessment or only the dominate vector as determined at step 208. The report may indicate the triggering TWA assessment event and which TWA measurements are determined to be clinically important based on the results of method 180 in FIG. 6. The report can include discrimination between discordant and concordant TWA and discrimination between depolarization/repolarization and repolarization-only alternans.

Reported information can further include a report of TWA trends and other physiological measurements or trends, such as heart rate, pacing rate, hemodynamic measures, mechanical alternans, etc. Physiological data that allows the correlation of TWA and other physiological signals or events is provided at step 238 for TWA report generation. An indication of the frequency of phase reversals in T-wave parameters measured on a beat-by-beat basis or other frequencies or indicators of TWA measurement contaminations (PVCs, T-wave artifact, etc.) may be reported as a measure of TWA consistency.

A TWA trend analysis may be performed at step 245 with a time-based graph of TWA measurements generated. Trend analysis allows a clinician to determine if TWA is a worsening condition which may indicate a worsening disease state. TWA trend analysis will incorporate other physiological parameters such as heart rate, heart rate variability, heart rate turbulence, arrhythmia incidence, and activity. The correlation between TWA trends and other physiological events may then be determined. After generating a TWA report and determining the TWA trend, method 200 may proceed to method 250.

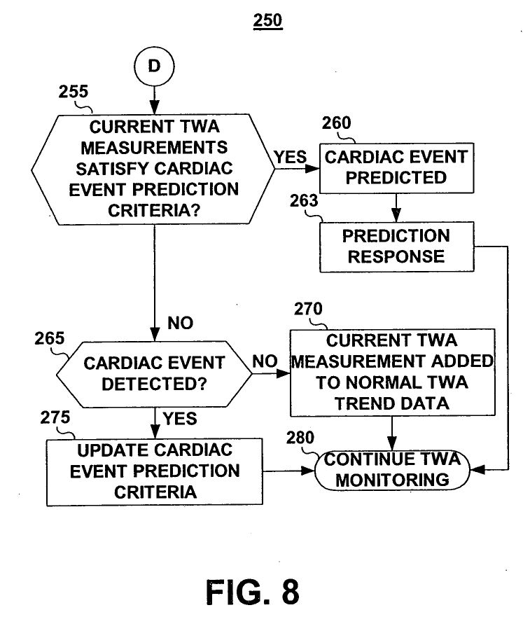

FIG. 8 is a flow chart summarizing a method 250 that may be used for applying TWA assessment results in managing therapies or predicting pathological cardiac events. At step 255, current TWA measurements are compared to a predefined cardiac event prediction threshold or other prediction criteria based on TWA assessment. One or more of the results generated for the TWA assessment report may be used at decision step 255. A cardiac event may be any pathologic event that is detectable by the IMD. A cardiac event may be an arrhythmia or a hemodynamic event. Numerous types of events may be detectable by the IMD based on physiological signals sensed by the IMD. Such events may include, for example, tachycardia or fibrillation, a change in blood pressure, a change in heart wall motion or heart chamber volume, or syncope, for example.

A multi-variate analysis may be performed for predicting a cardiac event at step 260. Multiple variables relating to TWA or other monitored physiological parameters may be relied upon by methods used for predicting a cardiac event, to promote higher sensitivity and specificity of cardiac event prediction than when using TWA criteria alone. For example, other criteria relating to blood pressure, heart rate or other physiological trends may be defined which must be satisfied, in addition to the presence of TWA predictive criteria.

If a cardiac event is predicted, a response to the prediction is provided at step 263. A cardiac event prediction response may include delivering a therapy, generating a patient warning, and/or generating a clinician warning to be stored in IMD 10 until the next device interrogation or transferred to a programmer/monitor. Delivered therapies may be therapies aimed at preventing the predicted cardiac event. For example, one response may include overdrive pacing the heart to prevent arrhythmias from occurring if the TWA occurs during a slow heart rate. Other therapy delivery responses may include neurostimulation or drug delivery to stabilize cardiac function. If a cardiac event is predicted based on a TWA measurement and a therapy is currently being delivered, the prediction response may include a withholding or adjustment of the therapy. For example, if extra-systolic stimulation is being delivered to achieve cardiac potentiation and TWA measurements satisfy cardiac event prediction criteria, the prediction response provided at step 263 may include deactivation of the stimulation therapy.

If the current TWA measurements do not meet prediction criteria at decision step 255, method 250 determines if any cardiac events are detected at decision step 265 based on monitored physiological signals. If a cardiac event occurs within a predefined time frame corresponding to TWA measurements, the TWA measurements are used to update the cardiac event prediction criteria at step 275 such that the current TWA measurements would have resulted in a positive prediction of a cardiac event. Through a learning process, prediction criteria can be updated based so that greater prediction accuracy may be achieved for future events.

If no cardiac events are detected at decision step 265, the prediction criteria are deemed reliable and no changes are made. The current TWA measurements are considered to be within a range that is not predictive of pathological cardiac events. At step 270, the current TWA measurements are added to the normal TWA trend data to update the normal range of TWA measurements.

After providing a prediction response (step 263), updating cardiac event prediction criteria (step 275) or adding current TWA measurements to normal TWA trend data (step 270), TWA monitoring continues at step 280. TWA assessment continues on a scheduled and/or triggered basis as described previously in conjunction with FIG. 3.

FIG. 9 is a flow chart summarizing a general method for detecting an alternans pattern in a physiological signal. Method 300 may be applied to EGM signals for detecting the presence of R-wave alternans or to mechanical cardiac signals for determining mechanical alternans. At step 301, the signal data to be evaluated is selected. The signal data has been stored previously (step 140 of method 100, FIG. 3) and is typically acquired simultaneously with EGM signal data used for measuring TWA to determine the association of TWA with other signal alternans. The signal data may be the same EGM signal used for measuring TWA, which may now be evaluated for measuring R-wave alternans. The signal data may alternatively be a different EGM signal acquired from a selected electrode configuration, using a sense amplifier adjusted for R-wave detection. The signal data selected at step 301 may be a physiological signal such as blood pressure or wall motion used for measuring mechanical alternans.

At step 305, signal conditioning techniques may be performed in order to improve the signal-to-noise ratio. At step 310, an A-B data matrix is generated by labeling the cardiac cycles in an alternating A-B pattern as described previously for the TWA measurement method. A signal parameter is measured for each of the cardiac cycles and stored accordingly in the A-B data matrix. At step 315, an alternans measurement is made by determining a beat-by-beat difference between parameter measurements obtained for “A” and “B” labeled cycles or by performing a spectral analysis on a time series stored in the A-B matrix. Computation of an alternans measurement may include averaging techniques.

At step 320, the consistency of the alternans measurement may be determined to ensure that signal artifact or other variations are not contributing to the alternans measurement. According to decision step 325 for evaluating the result of the alternans consistency determination and the magnitude of the alternans measurement relative to an alternans detection threshold criteria, alternans is either detected at step 335 or not detected at step 330. According to the present invention, the specific value utilized in step 325 as the alternans detection threshold is chosen as being somewhere within the range of approximately 30-50 uV. For example, according to an embodiment of the present invention, the alternans detection threshold is set equal to 36 uV so that T-wave alternans is detected in step 325 if the alternans measurement is determined to be consistent and the magnitude of the alternans measurement is greater than or equal to approximately 36 uV

Thus, a system and method have been described for providing TWA monitoring using signals acquired from an implanted electrode system. It is recognized that numerous variations of the embodiments described herein may be conceived for assessing TWA, generating a TWA report and using TWA assessment results for predicting cardiac events. The description and illustrated embodiments provided herein should therefore be considered exemplary, not limiting, with regard to the following claims.

What is claimed is:

1. A method of determining a cardiac event in a medical device, comprising:
acquiring a cardiac EGM signal from implanted electrodes;
defining a T-wave measurement window to be applied to the EGM signals relative to each cardiac cycle;
measuring a T-wave parameter within the T-wave measurement window for a plurality of cardiac cycles to generates a plurality of measured T-wave signals;
determining a T-wave alternans consistency in response to the plurality of measured T-wave signals; and
determining whether a T-wave signal of the plurality of measured T-wave signals is greater than a predetermined threshold.

2. The method of claim 1, wherein the predetermined threshold corresponds to a range between approximately 30 uV and 50 uV.

3. The method of claim 1, wherein the predetermined threshold is approximately equal to 36 uV.

4. The method of claim 1, wherein determining a T-wave alternans consistency comprises:
generating a matrix of the measured T-wave signals;
computing a T-wave alternans measurement from the generated matrix; and
determining the T-wave alternans consistency in response to the computed T-wave alternans measurement.

5. The method of claim 1, wherein acquiring the cardiac EGM signal comprises automatically adjusting a sense amplifier gain responsive to a voltage amplitude measured during a T-wave signal.

6. The method of claim 1, wherein defining the T-wave measurement window comprises measuring any of a QRS width, an S-T interval duration and a Q-T interval duration.

7. The method of claim 1, wherein the measured T-wave parameter is a T-wave signal voltage amplitude.

8. The method of claim 4, wherein generating a matrix of the T-wave parameter measurements, comprises:
labeling consecutive T-waves in an alternating “A-B-A-B” pattern, and
storing the T-wave parameter measurements made for the plurality of cardiac cycles according to the “A” or “B” label of the respective T-wave for which the T-wave parameter measurement was made.

9. The method of claim 8, wherein determining the T-wave alternans consistency comprises computing a difference between the “A” labeled T-wave parameter measurements and the “B” labeled T-wave parameter measurements.

10. The method of claim 1, wherein determining the T-wave alternans consistency comprises one of determining a frequency of phase reversals in differences computed between consecutive pairs of measured T-wave signals of the plurality of measured T-wave signals, determining the frequency of premature contractions in the acquired cardiac EGM signal, determining an effect of a respiration signal on the plurality of measured T-wave signals, and determining a frequency of T-wave signal artifacts in the acquired cardiac EGM signal.

11. The method of claim 1, further comprising:
measuring a heart rate associated with the plurality of measured T-wave signals; and
comparing the heart rate to a predetermined heart rate threshold.

12. The method of claim 1, further comprising:
determining a cardiac event in response to the determining a T-wave alternans consistency the determining whether a T-wave signal of the plurality of measured T-wave signals is greater than the predetermined threshold; and
performing one of controlling a preventative therapy and generating an alarm.

13. The method of claim 12 wherein controlling a preventive therapy corresponds to one of delivering overdrive pacing, delivering neurostimulation, delivering a drug, deactivating delivery of a therapy, controlling delivery of an extra systolic stimulation therapy, and adjusting a therapy delivery control parameter.

14. The method of claim 1, further comprising:
sensing a physiological signal;
determining a correlation between the physiological signal and the measurement of T-wave alternans.

15. A medical device, comprising:
a plurality of electrodes adapted for implantation in a patient's body for sensing cardiac EGM signals to sense a plurality of T-wave signals during a plurality of cardiac cycles; and
a microprocessor determining a T-wave alternans consistency in response to the plurality of T-wave signals and determining whether a T-wave signal of the plurality of T-wave signals is greater than a predetermined threshold.

16. The medical device of claim 15, wherein the predetermined threshold corresponds to a range between approximately 30 uV and 50 uV.

17. The medical device claim 15, wherein the predetermined threshold is approximately equal to 36 uV.
